(19) United States
(12) Patent Application Publication
(10) Pub. No.: US 2001/0049393 A1
(43) Pub. Date: Dec. 6, 2001

(54) Methods for defining MYC target genes and uses thereof

(75) Inventors: Hilary A. Coller, Seattle, WA (US); Todd R. Golub, Newton, MA (US); Carla Grandori, Seattle, WA (US); Pablo Tamayo, Cambridge, MA (US); Trenton Colbert, Seattle, WA (US); Robert Eisenman, Mercer Island, WA (US)

Correspondence Address: Doreen M. Hogle, Esq., HAMILTON, BROOK, SMITH & REYNOLDS, P.C., Two Militia Drive, Lexington, MA, 02421-4799, US

(73) Assignee: Whitehead Institute for Biomedical Research, Cambridge, MA (US)

(21) Appl. No.: 09/732,998

(22) Filed: Dec. 7, 2000

Related U.S. Application Data
(60) Provisional application No. 60/169,522, filed on Dec. 7, 1999.

(51) Int. Cl. A61K 31/138

(52) U.S. Cl. 514/651

(57) ABSTRACT

Identification of MYC target genes whose expression is either induced or repressed by c-myc induction in human fibroblasts is disclosed. Also disclosed are methods of inducing or repressing expression of MYC target genes.

### RELATED APPLICATION

This application claims the benefit of U.S. Provisional Application No. 60/169,522, filed on Dec. 7, 1999. The entire teachings of the above application is incorporated herein by reference.

### GOVERNMENT SUPPORT

The invention was supported, in whole or in part, by grant CA75125 from National Institutes of Health/National Cancer Institute. The Government has certain rights in the invention.

### BACKGROUND OF THE INVENTION

The c-myc protooncogene plays a key role in cell proliferation, differentiation, and apoptosis. c-myc transcripts are rapidly induced upon mitogenic stimulation and are down-regulated during cellular differentiation (Henriksson, M. and Luscher, B., 1996. Adv. Cancer Res. 68:109-182 1996; Amati et al., 1993. EMBO J. 13:5083-5087). Consistent with MYC's role in promoting cell proliferation, genetic alterations resulting in deregulation of myc expression are common to a wide range of tumor types (Magrath, I., 1990. Adv. Cancer Res. 55:133-270; Cole, M., 1986. Annu. Rev. Genet. 20:361-384).

MYC protein possesses a basic helix-loop-helix/leucine zipper domain that mediates dimerization with its partner, MAX. MYC-MAX heterodimers bind DNA at the E-box-related sequence, CACGTG, and other noncanonical sites, and activate transcription (Blackwood, E. et al., 1992. Curr. Opin. Genet. Dev. 2:227-235; Henriksson, M. and Luscher, B., 1996. Adv. Cancer Res. 68:109-182 1996). MYC has also been reported to repress transcription at specific initiator elements, although the mechanism involved has not been clarified (Li, L. et al., 1994. EMBO J. 13:4070-4079; Bush, A. et al., 1998. Genes Dev. 12:3797-3802).

Many previously reported MYC target genes are involved in metabolism and growth (Dang, C., 1999. Mol. Cell Biol. 19:1-11). The targets ornithine decarboxylase (Bello-Femandez, C. et al., 1993. Proc. Natl. Acad. Sci. USA. 90:7804-7808; Wagner, A. et al., 1993. Cell Growth Diff. 4:879-883), CAD (Miltenberger, R. et al., 1995. Mol. Cell. Biol. 15:2527-2535) and dihydrofolate reductase (Mai, S. and Jalava, A., 1994. Nucl. Acids Res. 22:2264-2273) suggest a role for MYC in DNA metabolism, while the targets ferritin and iron regulatory protein-2 suggest MYC may affect iron metabolism (Wu, K. et al., 1999. Science. 283:676-679). Previously reported targets involved with protein synthesis include the translation initiation factors EIF4E and 2A (Rosenwald, I. et al., 1993. Proc. Natl. Acad. Sci. USA. 90:6175-6178; Jones, R. et al., 1996. Mol. Cell. Biol. 16:4754-4764) and the RNA helicase MrDb (Grandori, C. et al., 1996. EMBO J. 15:4344-4357). Reported MYC targets that maybe critical for its effects on cell proliferation and immortalization include the phosphatase cdc25A (Galaktionov, K. et al., 1996. Nature. 382:511-517), and the catalytic subunit of telomerase (Wang, J. et al., 1998. Genes Dev. 12:1769-1774; Greenberg, R., et al., 1999. Oncogene. 18:1219-1226; Wu, K. et al., 1999. Oncogene. 18:1219-1226).

However, identifying additional MYC target genes by conventional methods has proven difficult. MYC-MAX heterodimers induce only a modest increase in transcription (Kretzner, L. et al., 1992. Nature. 359:426-429), and the short target recognition sequence provides little guidance for identifying additional target genes. Other available approaches for identifying MYC target genes to date have been time consuming, involving cDNA subtraction or isolation of MYC-MAX bound chromatin (Grandori, C. and Eisenman, R., 1997. Trends Biochem. Sci. 22:177-181).

### SUMMARY OF THE INVENTION

A new approach for identifying MYC target and a description of identified targets is described herein. Targets identified using this approach reinforce findings that MYC plays a role in cell transformation processes such as increased cell growth, proliferation and changes in cytoskeleton structure, as well as potential new role in cell differentiation, apoptosis DNA metabolism and functions associated with immunophilins.

MYC affects normal and neoplastic cell proliferation by altering gene expression, but the precise pathways remain unclear. As described herein, oligonucleotide microarray analysis of 6416 genes and ESTs was performed to determine changes in gene expression caused by induction of c-myc in primary human fibroblasts. In these experiments, 27 genes were consistently induced, and 9 genes were repressed. Pattern matching methods were also explored as described herein as an alternative approach for identifying MYC target genes. The genes that showed an expression profile most similar to endogenous c-myc in microarray-based expression profiling of myeloid differentiation models were highly enriched for the set of MYC target genes identified in the conditional myc induction experiments. Several targets identified herein suggest direct pathways for MYC function. Genes involved in cell growth include EIF5A, nucleolin and fibrillarin. A novel class of MYC targets are the immunophilins, including a 59 kDa FK506 binding protein, recently shown to localize to the mitotic spindle. Fibronectin, a critical protein for cell adhesion, was reproducibly down-regulated, while cytochrome C, a trigger for apoptosis, was up-regulated. MYC's functions in cell proliferation and immortalization are suggested by up-regulation of cyclin D2 and CksHs2, a cdk-binding protein, and down-regulation of the cdk inhibitor, p21^{Crp1}.

Thus, the invention relates to a method for inducing the expression of at least one of the following genes: AHCY, CCND2, ASS, FKBP52, PBEF, TRAP1, FABP52, GOS2, PPIF, hsRPB8, fibrillarin, TFRC, CksHs2, SLC16A1, IARS, HLA-DRB1, GRPE-homolog, GPI, HSPD1, HDGF, SF2, coup transcription factor, RPS11, EIF5A and EIF4γ, in a mammalian cell by inducing MYC transcriptional activation activity.

More specifically, induction of expression of these genes can occur where MYC expression is induced in the cell by transfecting or transducing the cell with a recombinant fusion gene that directs the expression of a chimeric receptor comprising MYC and a ligand binding domain and contacting the resulting cell with an appropriate ligand thereby inducing MYC expression. In a particular embodiment, the recombinant fusion gene directs the expression of a fusion protein containing MYC and the ligand binding domain of the estrogen receptor such that the ligand that induces c-myc is the estrogen analog 4-hydroxytamoxifen. In this embodiment, the ratio of the expression level observed in cells in the presence of ligand to the expression level observed in cells in the absence of ligand is preferably greater than 2. In this embodiment, induction can occur in a cell such that the cell is a primary human cell.

In another embodiment, the invention is directed to a method for repressing the expression of at least one of the following genes: AHCY, CCND2, ASS, FKBP52, PBEF, TRAP1, FABP52, GOS2, PPIF, hsRPB8, fibrillarin, TFRC, CksHs2, SLC16A1, LARS, HLA-DRB1, GRPE-homolog, GPI, HSPD1, HDGF, SF2, coup transcription factor, RPS11, EIF5A and EIF4γ in a mammalian cell by inhibiting MYC expression in said cell.

In another embodiment, the invention is directed to a method for causing transcriptional repression of at least one of the following genes: A2M, TPM1, PDGFRA, FN1, CTGF, COL3A1, CDKN1A and a dithiolethione-inducible gene in a mammalian cell by inducing MYC expression. In this embodiment, MYC expression is induced in the cell by transfecting or transducing the cell with a recombinant fusion gene which directs the expression of a chimeric receptor comprising MYC and a ligand binding domain and contacting the resulting cell with an appropriate ligand thereby inducing MYC expression. In a particular embodiment, the recombinant fusion gene directs the expression of a fusion protein containing MYC and the ligand binding domain of the estrogen receptor such that the ligand that induces c-myc is 4-hydroxytamoxifen. In this embodiment, the ratio of the expression level observed in cells in the presence of ligand to the expression level observed in cells in the absence of ligand is less than 0.5. In this embodiment, induction can occur in a cell such that the cell is a primary human cell.

In another embodiment, the invention is directed to a method for inducing at least one of the following genes: A2M, TPM1, PDGFRA, FN1, CTGF, COL3A1, CDKN1A and a dithiolethione-inducible gene in a mammalian cell by inhibiting MYC expression.

In another embodiment, the invention is directed to a method for identifying an agent that regulates MYC-dependent transcriptional regulation of gene expression including the steps of: producing an indicator cell that expresses a chimeric receptor comprising MYC and a ligand binding domain; contacting the resulting indicator cell with an appropriate ligand in the presence and absence of an agent to be evaluated for its ability to regulate MYC's transcriptional regulation activity; isolating mRNA from a plurality of indicator cells; and comparing the level of gene expression in the indicator cells in the presence or absence of the agent such that if the effect of MYC on the expression of the gene is enhanced or inhibited in the presence and not the absence of the agent, then the agent regulates MYC-dependent transcriptional regulation of gene expression. In one embodiment, the agent is tested for its ability to inhibit MYC-dependent transcriptional regulation of gene expression. In another embodiment, the agent is tested for its ability to activate MYC-dependent transcriptional regulation of gene expression. In a particular embodiment, the gene whose level of expression is being evaluated for regulation is one of the following: AHCY, CCND2, ASS, FKBP52, PBEF, TRAP1, FABP52, GOS2, PPIF, hsRPB8, fibrillarin, TFRC, CksHs2, SLC16A1, IARS, HLA-DRB1, GRPE-homolog, GPI, HSPD1, HDGF, SF2, coup transcription factor, RPS11, EIF5A and EIF4γ, A2M, TPM1, PDGFRA, FN1, CTGF, COL3A1, CDKN1A and a dithiolethione-inducible gene. In this embodiment, the chimeric receptor can be a fusion containing MYC and the ligand binding domain of the estrogen receptor such that the ligand that induces c-myc is the estrogen analog 4-hydroxytamoxifen. In this embodiment, the agent can be evaluated in the presence of cycloheximide. In this embodiment, the level of gene expression can be determined by hybridization to an oligonucleotide microarray. Alternatively, the level of gene expression can be determined by Northern blot analysis.

In another embodiment, the invention is directed to a method for treating cell proliferative disorders by altering the transcriptional regulatory activity of MYC in cells. In a particular embodiment, the cells are hematopoietic cells.

In another embodiment, the invention is directed to a method for treating cell proliferative disorders by altering MYC expression in cells. In a particular embodiment, the cells are hematopoietic cells.

In another embodiment, the invention is directed to a method for detecting cell proliferative disorders including the steps of: isolating a cell of interest; determining the level of expression of at least one gene that is regulated by MYC; and comparing the level of expression in the cell of interest and cells that are not characterized as having a proliferative disorder of the gene such that altered expression of the gene is indicative of a proliferative disorder. The isolated cell can be a hematopoietic cell. In this embodiment, the gene that is regulated by MYC can be one of the following: AHCY, CCND2, ASS, FKBP52, PBEF, TRAP1, FABP52, GOS2, PPIF, hsRPB8, fibrillarin, TFRC, CksHs2, SLC16A1, LARS, HLA-DRB1, GRPE-homolog, GPI, HSPD1, HDGF, SF2, coup transcription factor, RPS11, EIF5A and EIF4γ, A2M, TPM1, PDGFRA, FN1, CTGF, COL3A1, CDKN1A and a dithiolethione-inducible gene.

In another embodiment, the invention is directed to a method for evaluating antiproliferative drug candidates including the steps of: contacting a cell that conditionally expresses MYC with the anti-proliferative drug candidate; inducing MYC expression; isolating mRNA from the cell; and comparing the level of gene expression of at least one MYC-regulated gene in cells in the presence or absence of the anti-proliferative drug candidate such that a difference in expression indicates the effect of the anti-proliferative drug candidate on the transcriptional regulatory activity of MYC. In a particular embodiment, the anti-proliferative drug candidate is evaluated in hematopoietic cells.

In another embodiment, the present invention is directed to a method for detecting MYC target genes comprising the steps of: inducing MYC expression in an indicator cell; isolating mRNA from induced indicator cells; and comparing the level of gene expression of at least one mRNA transcript in cells induced for MYC expression with the level of gene expression of the mRNA transcript in cells that have not been induced for MYC expression, such that altered expression of the gene corresponding to the mRNA transcript in MYC-induced cells indicates the gene is a MYC target gene. In a particular embodiment, the level of gene expression is determined using a hybridization assay. The hybridization assay can include a step of contacting cellular mRNA with an oligonucleotide microarray fused to a chip. The chip can be one of the following: Affymetrix HUM6000-1, Affymetrix HUM6000-2, Affymetrix HUM6000-3 and Affymetrix HUM6000-4.

In another embodiment, the invention is directed to a method for inducing the expression of at least one of the following genes that is directly induced by MYC: AHCY, CCND2, ASS, FKBP52, TRAP1, FABP52, GOS2, PPIF, fibrillarin, TFRC, CksHs2, SLC16A1, IARS, GRPE-homolog, HDGF, and EIF5A in a mammalian cell comprising inducing MYC expression in said cell.

In another embodiment, the invention is directed to a method for causing transcriptional repression of at least one of the following genes that is directly repressed by MYC: A2M, TPM1, PDGFRA, FN1, CTGF, COL3A1, and CDKN1A in a mammalian cell comprising inducing MYC expression.

### BRIEF DESCRIPTION OF THE DRAWINGS

FIGS. 1A-1C are scatter plots showing the expression level of individual genes in two experiments. For each gene, the RNA expression level in one sample is given on the x axis and the expression level for the same gene in the other sample on the y axis.

FIG. 1A shows expression levels for control RNA prepared from two independent samples and provides a separate demonstration of variability from target preparation and scanning.

FIG. 1B shows expression levels from two different samples. The plot demonstrates variability in expression levels attributed to independent sampling.

FIG. 1C shows gene expression levels for RNA prepared from MER infected 4OHT-treated fibroblasts and control-infected 4OHT-treated fibroblasts converted into target and hybridized. The plot further demonstrates expression levels observed for putative MYC target genes.

FIG. 1D shows expression levels for control RNA prepared from human leukemia cells exposed to one or two rounds of polyA selection, converted into target and hybridized to oligonucleotide arrays. The plot demonstrates variability in gene expression levels attributed to target preparation and scanning.

FIG. 1E shows expression levels for control RNA prepared from two independent samples of proliferating human fibroblasts (e.g., CCL-153 (American Type Culture Collection)), converted into target and hybridized. The plot demonstrates variability in gene expression attributed to independent sampling.

FIG. 1F shows expression levels for RNA prepared from MYC-ER-infected OHT treated fibroblasts and control-infected OHT-treated fibroblasts converted into target and hybridized. The plot demonstrates the expression levels observed for putative MYC target genes.

FIGS. 2A and 2B are Venn diagrams of the number of genes altered in each of three independent MYC-ER experiments. FIG. 2A summarizes the number of putative MYC target genes which are induced in response to conditional MYC activation. FIG. 2B summarizes the number of target genes repressed by MYC activation.

FIGS. 3A-3C are Northern blots of putative MYC target genes.

FIG. 3A is a Northern blot utilizing RNA harvested from the indicated control, or MYC-ER expressing fibroblast, assayed in the presence or absence of 4OHT as indicated. The fibroblasts expressing MYC-ER Δ-MER were transduced with a deletion mutant of the MYC-ER fusion protein incapable of transactivating MYC-responsive genes. Ethidium bromide-stained rRNA levels demonstrates similar loading in each lane.

FIG. 3B is a Northern blot of samples from a MYC-ER conditional induction experiment showing induction of EIF5A and cyclin D2 genes. Induction conditions are given in the text.

FIG. 3C is a Northern blot of samples from a MYC-ER conditional induction experiment showing repression of p21 transcript levels after MYC-ER induction.

FIG. 4 is a schematic representation of MYC target genes within a cell. Depicted is a selection of the MYC targets identified herein along with their subcellular localization.

FIG. 5 is a schematic representation of the expression profiles of genes that were identified as behaving most similarly to an induced myc target a constitutive MYC overexpression experiment and in a hematopoietic cell differentiation system.

FIG. 6 is a table listing the 27 genes activated by MYC and the 9 genes repressed by MYC. Relative activation and repression levels are shown.

### DETAILED DESCRIPTION OF THE INVENTION

The c-myc protooncogene, originally identified as the cellular homolog of the v-myc ontogeny present in retroviruses (Bishop, J., 1983. Ann. Rev. Biochem. 52:301-354), has been shown to play a key role in cell proliferation based on the effects of its overexpression and underexpression, its expression pattern and its association with tumors. As used herein, “c-myc” refers to the cellular version of the gene; “v-myc” refers to the viral version of the gene. A “gene,” as is generally understood and used herein, refers to a DNA sequence encoding a single polypeptide chain or protein, and, as used herein, can include untranslated regions such as those at the 5′ and 3′ ends of the coding sequence. Activation of myc with chimeric proteins induces cell cycle entry in quiescent cells (Eilers, M. et al., 1989. Nature. 340:66-68; Eilers, M. et al., 1991. EMBO J. 10:133-141). Constitutive c-myc expression also potentiates S phase entry (Kaczmarek, L. et al, 1985. Science. 228:1313-1315), shortens G1 (Kam, J. et al., 1989. Oncogene. 4:773-787), reduces growth factor requirements (Armelin, H. et al., 1984. Nature. 310:655-660), inhibits differentiation (Coppola, J. and Cole, M., 1986. Nature. 320:760-763) and prevents cells from leaving the cell cycle (Freytag, S., 1988. Mol. Cell. Biol. 8:1614-1624). As used herein, “expression” refers to the process that results in the production of a protein product encoded by a specific gene. Gene expression can be “induced” (as used herein, “induced” refers to expression that occurs in response to a specific “induction signal,” usually a small molecule or transcription activator).

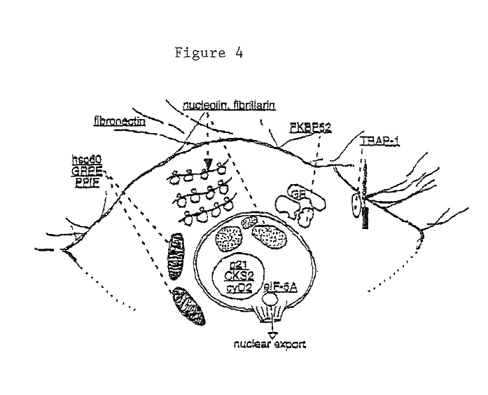

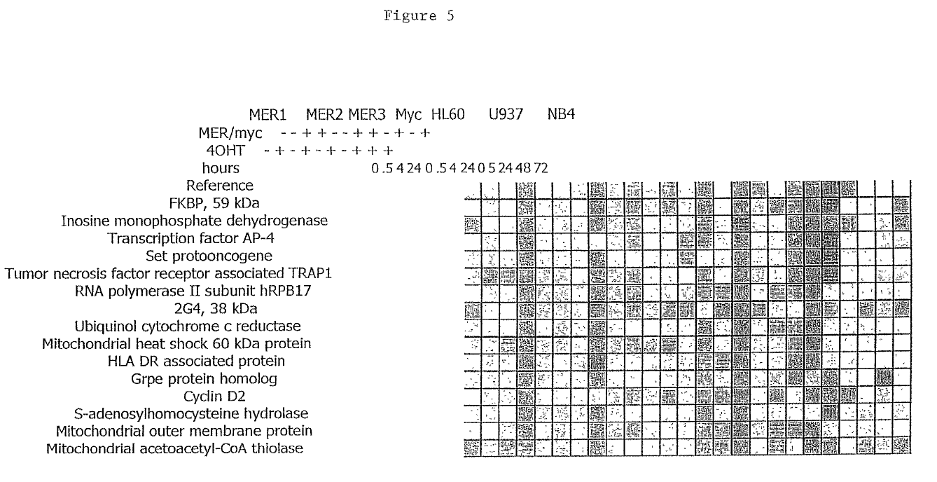

It is likely that MYC functions as a regulator of transcription and interacts with many upstream and downstream factors in order to produce effects on so many processes (see FIG. 4); by regulating expression of downstream genes that are involved in various cellular functions, a broad range of functions are affected by the precise expression levels of MYC. As used herein, “MYC” refers to the protein product of a myc gene. As used herein, “upstream” refers to factors and events that regulate MYC expression, whereas “downstream” refers to factors and events that are regulated by MYC. As used herein, downstream factors that are transcriptionally regulated by MYC are referred to as “targets.” As used herein, “transcriptional regulation” refers to altered gene expression; “activators” increase transcription and “repressors” decrease transcription of specific targets.

Many signal transduction pathways utilize a series of factors to regulate specific cellular processes. Many of these factors are used in more than one pathway. MYC is likely a factor that relays messages from upstream signals to effect downstream changes. A method for producing such effects is through transcriptional regulation and the factors that regulate transcription of genes are referred to as “transcription factors.” Thus, although MYC is involved in diverse pathways affecting many cellular processes, these effects only manifest themselves with the aid of downstream “effector genes” or transcription factors which, in turn, regulate effector genes or transcription factors further downstream. As used herein, “effector genes” refer to targets that are directly responsible for effecting a specific cellular process. Many questions regarding the exact role of MYC in signaling pathways can be addressed by identifying downstream MYC targets. Such an identification is described herein.

Described herein is a method for identifying downstream targets regulated by MYC. The method of the present invention is directed to altering MYC expression, thereby altering the expression of downstream target genes. Activation or repression of MYC expression will lead to altered expression of downstream target genes. That is, induction of MYC expression will induce expression of genes that are activated by MYC and inhibit the expression of genes that are repressed by MYC. Conversely, repression of MYC will inhibit expression of genes that are activated by MYC and activate expression of genes that are repressed by MYC. Detecting the levels of expression of a gene or several genes with and without altered MYC expression thus detects target genes that exhibit altered expression in response to altered MYC expression. Alteration of MYC expression can occur in a number of ways that will be readily recognized by the skilled artisan. Additionally, the method described herein could easily be adapted for use in various cell types and in various cell types in different stages of the cell cycle.

A hybridization assay is described herein wherein changes in RNA expression after alteration of MYC expression detects MYC target genes. RNA transcripts are obtained from cells, either in vivo or ex vivo, and assayed for altered expression by hybridizing the mRNA to oligonucleotides which are representative of one or more cellular genes. This strategy involves altering MYC expression and then monitoring the expression of other genes are affected. The alteration of MYC expression can either be a repression of MYC expression, wherein cellular levels of MYC are lower than in the unrepressed state, or an induction of MYC expression, wherein cellular levels of MYC are higher than than in the uninduced state. An example is provided wherein MYC expression is induced in cells prior to extracting RNA. Expression levels in MYC-induced cells is compared to expression levels in uninduced cells. Differences in expression levels between induced and uninduced cells indicate MYC target genes.

One induction strategy can be utilization of a fusion protein that has MYC fused to the ligand binding domain of a receptor. Preferred receptors are those that translocate into the cellular nucleus in response to an external signal, e.g., hormone receptors. The ligand-binding domains of suitable receptors can be identified using methods known in the art. In the example presented herein, induction of MYC was effected using a fusion protein (“MYC-ER”) that has MYC joined to the ligand binding domain of the estrogen receptor (Eilers, M. et al., 1989. Nature. 340:66-68; Eilers, M. et al., 1991. EMBO J. 10:133-141; Littlewood, T. et al., 1995. Nucl. Acids Res. 23:1686-1690). The steroid receptor fusion molecule does not activate c-myc transcription until the ligand binding domain of the fusion protein is bound to a corresponding ligand such as the estrogen analog, 4-hydroxytamoxifen (OHT). As used herein, “corresponding ligand” refers to the binding partner of a receptor such that a particular function of the receptor is effected. Thus, by introducing OHT into cell media in tissue culture or in animals, MYC-ER can be activated to induce c-myc.

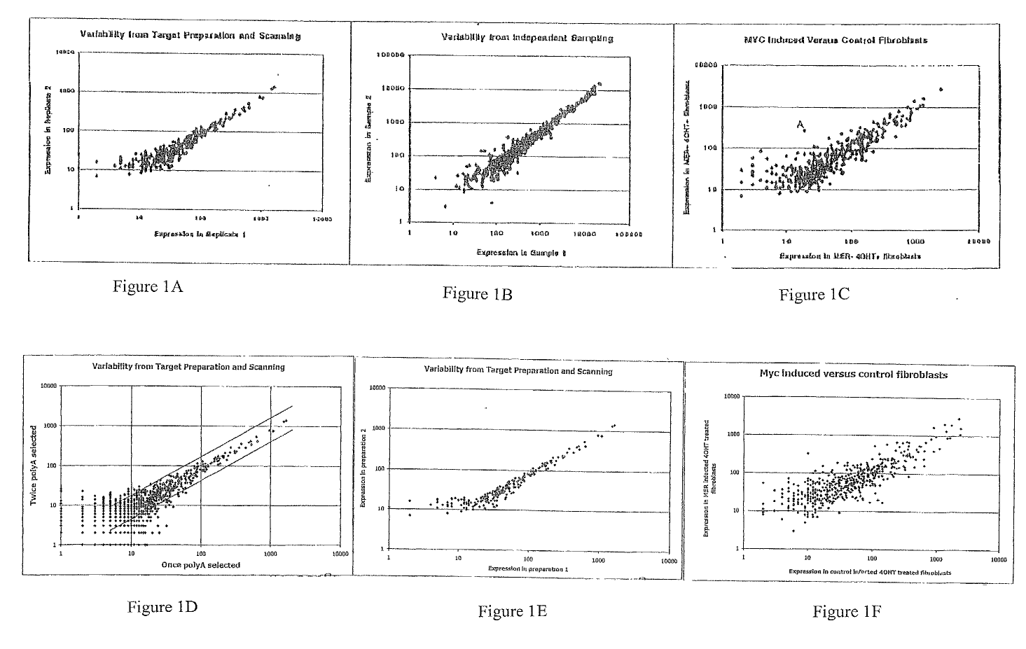

Expression of other genes was monitored using a hybridization assay. This assay involves isolating total cellular mRNA and hybridizing the mRNA to oligonucleotide microarrays fused to the surface of chips. The cellular mRNA is isolated from cells that are induced or uninduced for MYC expression (see FIG. 1). The oligonucleotide microarrays contain short sequences from a library of known genes. Thus, a measure of mRNA hybridization to oligonucleotide microarrays provides a measure of the proportion of any particular mRNA relative to the total mRNA. Since mRNA transcripts are the products of gene expression, an increase in the proportion of mRNA transcript from a particular gene relative to the total cellular mRNA indicates that the particular gene has been activated. Conversely, a decrease indicates the gene is repressed. Specifically, the hybridization assay utilized arrays that allowed for monitoring of 6416 human genes and unnamed ESTs as potential MYC targets. Chips containing microarrays with a different representation of cellular genes can also be used to identify additional MYC targets.

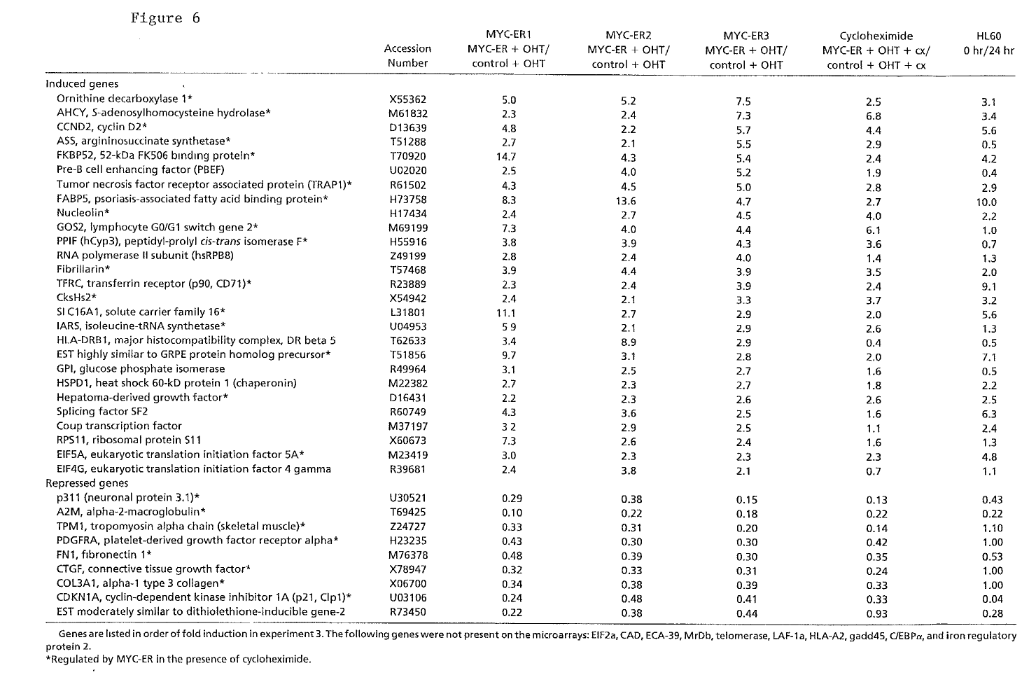

Using oligonucleotide microarrays to monitor the effects of induced MYC expression, 27 target genes were found that are activated by MYC and 9 target genes were found that are repressed by MYC (see FIG. 6). Based on changes in expression in the presence of cycloheximide, it was determined that most MYC target genes (18/27 of activated targets and 8/9 for repressed targets) are “direct targets,” used herein to refer to target genes that are directly regulated by MYC and not by an intermediate transcription factor. This finding, coupled with the observation that none of the putative MYC target genes identified are transcription factors, argues against the idea that MYC's role is to activate a transcriptional cascade. Thus, the genes regulated by MYC are likely to be effector genes whose activities lead directly to specific cellular function.

The results of previous studies along with target genes identified by the method described herein, suggest a role for MYC in regulating processes associated with cell transformation (increases in cell size, cell division even in the absence of mitogenic stimuli, alterations in cell adhesion, and changes in the shape and organization of the cytoskeleton) as well as roles in cellular differentiation, apoptosis, DNA metabolism, protein folding, and processes associated with immunophilins. Described herein are genes regulated by MYC that affect cell size and shape (e.g., ornithine decarboxylase; argininosuccinate synthetase, hereinafter, “ASS;” nucleolin; an RNA polymerase II subunit, hereinafter, “hsRPB8;” fibrillarin; isoleucine-tRNA synthetase, hereinafter, “LARS;” splicing factor 2, hereinafter, “SF2;” ribosomal protein 11, hereinafter, “RPS 11;” eukaryotic translation initiation factors SA and 4γ, hereinafter, “EIF5A and “EIF4γ,” respectively; tropomyosin alpha chain, hereinafter, “TPM1;” fibronectin 1, hereinafter, “FN1;” connective tissue growth factor, hereinafter, “CTGF;” and alpha-1 type 3 collagen, hereinafter, “COL3A1”). Also described herein are genes that affect cell proliferation (e.g., cyclin D2, hereinafter, “CCND2;” pre-B cell enhancing factor, hereinafter, “PBEF;” psoriasis-associated fatty acid binding protein, hereinafter, “FABP5;” nucleolin; lymphocyte G0/G1 switch gene 2, hereinafter, “G1S2;” fibrillarin; CksHs2; hepatoma-derived growth factor, hereinafter, “HDGF;” SF2; coup transcription factor; RPS11; platelet-derived growth factor receptor alpha, hereinafter, “PDGFRA;” CTGF; and cyclin-dependent kinase inhibitor 1A, hereinafter, “CDKN1A”). Also, as described herein, the method of the present invention identified effector genes involved in apoptosis (e.g., tumor necrosis factor receptor associated protein, hereinafter, “TRAP1”), metabolism (e.g., ornithine decarboxylase; S-adenosylhomocysteine hydrolase, hereinafter, “AHCY;” ASS; transferrin receptor, hereinafter, “TFRC;” a member of the solute carrier family 16, hereinafter, “SLC16A1;” and glucose phosphate isomerase, hereinafter, “GPI”), and protein folding (e.g., an EST similar to GRPE protein homolog precursor, hereinafter, “GRPE-homolog;” heat shock 60 kDa protein 1, hereinafter, “HSPD1;” and alpha-2-macroglobulin, hereinafter, “A2M”). Additionally, using the method of the present invention, members of the immunophilin family of proteins were identified as MYC targets (e.g., the 52-kDa FK506 binding protein, hereinafter, “FKBP52;” and peptidyl-prolyl cis-trans isomerase, hereinafter, “PPIF”). Two genes were identified that have yet to be characterized fully, neuronal protein 3.1 (hereinafter, “p311”) and an EST similar to dithiolethione-inducible gene-2. One target was identified that has been associated with rheumatoid arthritis and idiopahtic nephrotic syndrome (major histocompatibility complex DR beta 5, hereinafter, “HLA-DRB1”).

A major effect of MYC on both Drosophila and mammalian cells is to increase the accumulation of cell size (Johnston, L. et al., 1999. Cell. 98:779-790; Iritani, B. and Eisenman, R., 1999. Proc. Natl. Acad. Sci. USA. 96:13180-13185). Data described herein provide support for the view that MYC directly influences cell size through protein synthesis. Earlier work had indicated that the rate-limiting translational initiation factor, EIF4E, is induced by MYC (Rosenwald, I. et al., 1993. Proc. Natl. Acad. Sci. USA. 90:6175-6178). Work described herein indicates that MYC induces EIF5A, a translation initiation factor also thought to be involved in nucleocytoplasmic transport (Rosorius, O. et al., 1999. J. Cell Sci. 112:2369-2380; Elfgang, C. et al., 1999. Proc. Natl. Acad. Sci. USA. 96:6229-6234). Interestingly, MYC leads to increased levels of the previously identified target ornithine decarboxylase (Bello-Femandez, C. et al., 1993. Proc. Natl. Acad. Sci. USA. 90:7804-7808; Wagner, A. et al., 1993. Cell Growth Diff 4:879-883; FIG. 6), which regulates a hypusine modification of EIF5A that is critical for its function (Park, M. et al., 1998. J. Biol. Chem. 273:1677-1683). Other cell-size associated genes identified as MYC targets herein include several genes involved in nucleolar rRNA processing such as the structural proteins fibrillarin and nucleolin, the ribosomal protein RPS11, and EIF4γ.

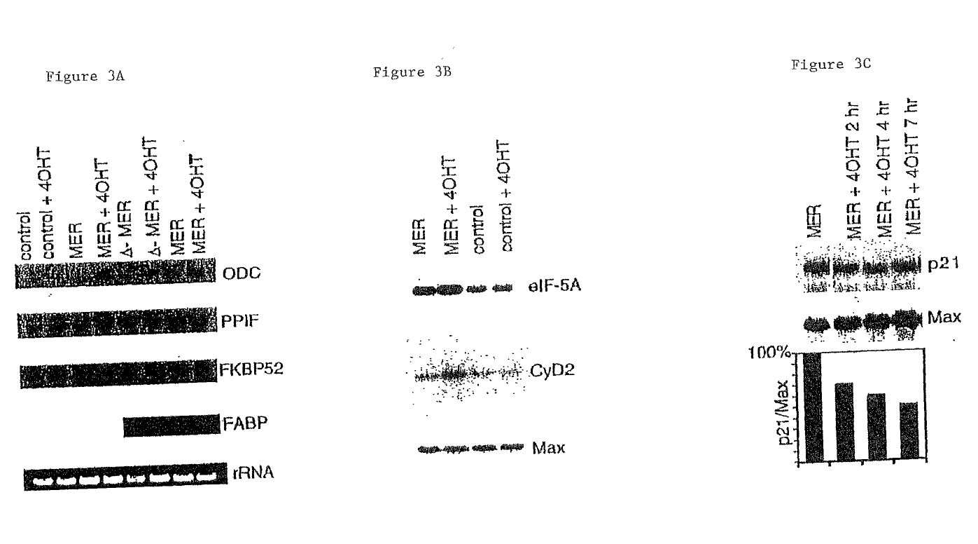

MYC had previously been implicated in having a role in cell cycle progression, and, thus, cell proliferation. Earlier studies reported that MYC decreases the amount of the cdk inhibitor, p27^{KIP1}, bound to cyclin E/cdk2 complexes (Vlach, J. et al., 1996. EMBO J. 15:6595-6604; Muller, D. et al., 1997. Oncogene. 15:2561-2576; Perez-Roger, I. et al., 1997. Oncogene. 14:2373-2381). The results presented herein suggest novel interactions between MYC and the cell cycle machinery and confirm previously characterized interactions. For example, the identification herein of CCND2 as a direct MYC target gene is consistent with other recent reports (Perez-Roger, I. et al., 1997. Oncogene. 14:2373-2381). CCND2 may contribute to cell proliferation by directly increasing phosphorylation of the retinoblastoma protein via its association with cdk4, or by sequestering p27^{KIP1} (Polyak, K. et al., 1994. Genes Dev. 8:9-22; Sherr, C. and Roberts, J., 1995. Genes Dev. 9:1149-1163). Also described herein is the fact that MYC induces CksHs2, a homolog of the yeast proteins CKS and p13^{suc1}, essential proteins that bind tightly to some cdk's, and play a role in cell viability and proliferation (Hayles, J. et al., 1986. Mol. Gen. Genet. 202:291-293; Hadwiger, J. et al., 1989. Mol. Cell. Biol. 9:2034-2041; Hindley, J. et al., 1987. Mol. Cell. Biol. 7:504-511). In addition, MYC is shown herein to repress expression of the CDKN1A (Harper, J. et al., 1993. Cell. 75:805-816; Xiong, Y. et al., 1993. Nature. 366:701-704; FIG. 3C). Decreased CDKN1A activity may represent another mechanism by which MYC increases cdk activity and cell proliferation.

A connection between MYC and cell adhesion is suggested by the observed repression of the extracellular matrix proteins, FN1 and COL3A1. Repression of both of these proteins has been reported to accompany cell transformation, and their loss may contribute to the decreased adhesiveness and a more rounded cell shape observed in transformed cells (Olden, K. and Yamada, K., 1977. Cell. 11:957-969). The finding that MYC represses transcription of the actin binding protein, TPM1, also provides a potential link between MYC overexpression and the cytoskeletal dysregulation commonly observed in transformed cells. TPM1 repression is a common change accompanying neoplastic transformation (Cooper, H. et al., 1985. Mol. Cell. Biol. 5:972-983); overexpression of tropomyosin can abolish a transformed phenotype (Prasad, G. et al., 1993. Proc. Natl. Acad. Sci. USA. 90:7039-7043); and antisense-induced reduction in tropomyosin levels confer anchorage independent growth potential (Boyd, J. et al., 1995. Proc. Natl. Acad. Sci. USA. 92:11534-11538).

Another physiological hallmark of MYC overexpressing cells is high levels of apoptosis. TRAP1 binds to the intracellular domain of the tumor necrosis factor receptor (Song, H. et al., 1995. J. Biol. Chem. 270:3574-3581), is a direct MYC target, and may be part of a general pathway for increased apoptosis in cells overexpressing MYC, as well as the mechanism by which MYC causes elevated susceptibility to TNF-α mediated apoptosis (Klefstrom, J., et al. 1994. EMBO J. 13:5442-5450).

Previous reports and genes identified herein suggest MYC target genes are involved in metabolism (Dang, C., 1999. Mol. Cell. Biol. 19:1-11). The targets ornithine decarboxylase (Bello-Fernandez, C. et al., 1993. Proc. Natl. Acad. Sci. USA. 90:7804-7808; Wagner, A. et al., 1993. Cell Growth Diff 4:879-883), CAD (Miltenberger, R. et al., 1995. Mol. Cell. Biol. 15:2527-2535) and dihydrofolate reductase (Mai, S. and Jalava, A., 1994. Nucl. Acids Res. 22:2264-2273) suggest a role for MYC in DNA metabolism, while the targets ferritin and iron regulatory protein-2 (Wu, K. et al., 1999. Science. 283:676-679) and TFRC described herein, suggest MYC may affect iron metabolism. Additional MYC targets identified herein support MYC's role in metabolism. These targets include AHCY, ASS, SLC16A1 and GPI.

The method of the present invention identified MYC targets that suggest a regulatory role for MYC in protein folding. MYC consistently activated gene encoding a protein highly similar to GRPE, the GRPE-homolog, as well as HSPD1 and TRAP 1, which is homologous to the heat shock 90 kDa protein. MYC is also shown herein to repress A2M expression, which has been implicated as being responsible for increased aggregation in Alzheimer's disease.

It is also shown herein that MYC regulates a previously unsuspected target class of proteins: the immunophilins. Two immunophilins, PPIF and FKBP52, are identified as direct MYC targets. FKBP52 forms a multimeric complex with steroid receptors and has been localized to the mitotic spindles (Perrot-Applanat, M. et al., 1995. J. Cell Sci. 108:2037-2051). Mutants of FKBP52 in Arabidopsis showed defects in cell proliferation in response to steroid signals (Sanchez, E., 1990. J. Biol. Chem. 265:22067-22070; Ning, Y. and Sanchez, E., 1993. J. Biol. Chem. 268:6073-6076; Vittorioso, P. et al., 1998. Mol. Cell. Biol. 18:3034-3043).

Defects in MYC targets result in a wide range of diseases and disorders. Defects in control of cell cycle and proliferation, referred to hereinafter as “proliferative disorders,” are characterized by tumor growth, cancer and psoriasis, whereas defects in other MYC targets have been implicated in neural tube defects, Alzheimer's disease, rheumatoid arthritis, idiopathic nephrotic syndrome, cystathionine beta-synthase deficiency, methionine adenosyltransferase deficiency and citrullinemia. Methods are described herein that lead to the regulation of genes responsible for these disorders and thus serve as methods potentially useful in therapeutic treatment of these and other disorders associated with MYC-regulated targets.

The invention will be further illustrated by the following nonlimiting examples.

### EXAMPLES

### Materials AND Methods

The following methods and materials were used in the work described herein:

Retroviral Vectors and Cell Culture

Amphotropic viral stocks were generated by co-transfection of pBabe-puro plasmid containing MYC-ER™ or Δ-MYC-ER™ (Littlewood, T. et al., 1995. Nucl. Acids Res. 23:1686-1690) together with Psi⁻helper construct (Muller, A. et al., 1991. Mol. Cell. Biol. 11:1785-1792) in 293T cells. Subconfluent WI38 cells (ATCC cat #CCL75) grown in DMEM with 10% FCS were infected with 5 mL of viral supernatant on two consecutive days. The next day, cells were plated at ˜10⁴cells/cm² in phenol-red free DME medium with 10% FCS, and selected in the presence of puromycin for pBABE vectors. Cells were grown to confluence, for seven to eight days, without media changes. Density arrested cells were induced with 200 nM OHT (4-hydroxy-tamoxifen) or serum starved (0.1% FCS) for 48 hours and then induced. Where specified, cells were exposed to cycloheximide (10 micrograms/mL) for 30 minutes prior to addition of OHT.

High Density Oligonucleotide Array Expression Analysis

A complete protocol for converting RNA into “target” suitable for hybridization to microarrays is available at web site http://www.genome.wi.mit.edu/MPR. Briefly, polyA mRNA was selected with oligo-dT beads from total RNA extracted with Trizol reagent (Life Technologies, Gaithersburg, Md.), and used to create cDNA with a T7-polyT primer and the reverse transcriptase Superscript II (Gibco-BRL, Gaithersburg, Md.). Approximately 1 microgram of cDNA was subjected to in vitro transcription in the presence of biotinylated UTP and CTP. Target for hybridization was prepared by combining 40 micrograms of fragmented transcripts with sonicated herring sperm DNA (0.1 mg/mL) and 5 nM control oligonucleotide in a buffer containing 1.0 M NaCl, 10 mM Tris-HCl (pH 7.6) and 0.005% Triton X-100. Target was hybridized for 16 hours at 40° C. to a set of four oligonucleotide arrays (HUM6000-1, HUM6000-2, HUM6000-3, HUM6000-4; Affymetrix, Santa Clara, Calif.) containing probes for 6416 human genes (5223 known human genes and 1193 unnamed ESTs). Arrays were washed at 50° C. with 6×SSPET (0.9 M NaCl, 60 mM NaH₂O₄, 6 mM EDTA, 0.005% Triton X-100, pH 7.6), then at 40° C. with 0.5×SSPET. Arrays were then stained with streptavidin-phycoerythrein. Fluorescence intensities were captured with a laser confocal scanner (Affymetrix, Santa Clara, Calif.) and the Genechip software (Affymetrix, Santa Clara, Calif.).

Expression data were analyzed as described previously (Tamayo, P. et al., 1999. Proc. Natl. Acad. Sci. USA. 96:2907-2912), including thresholding small and negative expression values to 20. Genes most similar to MYC were identified in the myeloid differentiation experiments based on a Euclidean distance metric, after eliminating genes that failed to vary in expression level within an experiment by a factor of three and an absolute value of 100, and normalizing within experiments to a mean of zero and a standard deviation of 1.

Analysis of RNA by Northern Blots

Northern blots were performed according to standard procedures (Ausubel, F. et al., 1990. Current Protocols in Molecular Biology. Wiley Interscience, New York). For cyclin D2 and p21, complete cDNA was used as probes. For FKBP52, a PCR amplicon of bps 1215-1767 (accession number M88279) was used; for FABP5 (PA-FA-BP), bps 60-481 (M94856); for ODC1, bps 1198-1984 (X55362); for PPIF (hCyP3), bps 404-803 (M80254); and for EIF5A, bps 46-512 (U17969). To assess the relative amounts of RNA loaded into each lane, the same filter was stripped and hybridized with a PCR product for GAPDH or MAX, genes that remain essentially constant among the samples. Hybridized filters were exposed sequentially to x-ray films and PhosphorImager screens.

### Example 1

MYC Targets Identified with MYC-ER: Introduction of the MYC-ER Gene into Human Fibroblasts by Retroviral Transduction

Treatment of the transduced cells with OHT, caused 20% of the cells to enter the cell cycle by 17 hours. In contrast, only 1-6% of, OHT-treated, non-MYC-ER expressing controls ever enter S phase. Hyperphosphorylation of Rb, activation of Cdk2, and increases in transcript levels of three known MYC target genes: MrDb (Grandori, C and Eisenman, R., 1997. Trends Biochem. Sci. 22:177-181), ornithine decarboxylase (Bello-Fernandez, C. et al., 1993. Proc. Natl. Acad. Sci. USA. 90:7804-7808; Wagner, A. et al., 1993. Cell Growth Diff. 4:879-883), and cdc25A (Galaktionov, K. et al., 1996. Nature. 382:511-517), are observed within 5 hours following OHT treatment. In three separate microarray experiments, ODC levels increased 5 to 7.5-fold.

In addition, MYC-ER stimulated cells eventually undergo apoptosis 48 to 72 hours after serum withdrawal. For microarray analysis, RNA was harvested from these cells 9 hours after OHT treatment, based on the reasoning that direct MYC targets would have increased or decreased in expression by this time, yet the many other downstream effects that occur as cells enter S phase at 17 hours would be minimized.

It was first determined whether the “signal,” in terms of changes in RNA levels caused by MYC induction, is greater than the background “noise” of fluctuations in gene expression expected from experimental variables. MYC activation of fibroblasts, as depicted in FIG. 1, resulted in a larger number of genes showing a given change in expression level as compared with the variability observed from target preparation and independent samplings of the same cell line (see FIG. 1). Based on the observation that few genes changed expression level by more than two-fold in the control experiments (˜2 per 1000 for technical variability and ˜20 per 1000 for biological variability in primary human fibroblasts), a threshold of a two-fold change in expression level between MYC-ER infected, OHT-stimulated samples and empty virus-infected, OHT-treated controls was adopted for identifying putative MYC targets.

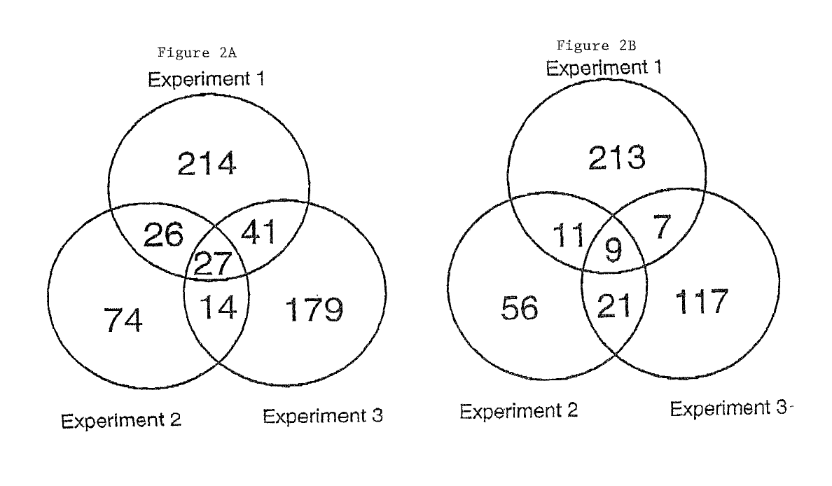

Conditional MYC induction was performed in three independent experiments. Shown in FIG. 2 are Venn Diagrams representing the number of genes that changed expression levels by at least two-fold in each of the three experiments, and the overlap among the experiments.

The criteria for increased gene expression were as follows: (1) the gene was called “present” in the MYC-ER+OHT sample; (2) the ratio of the expression level in the MYC-ER+OHT sample to the expression level in the control+OHT sample was greater than 2; and (3) the ratio of control+OHT to control was not greater than two.

The criteria for decreased (e.g., repressed) gene expression were as follows: (1) the gene was called “present” in the control+OHT sample; (2) the ratio of expression level in the MYC-ER+OHT sample to the expression level in the control+OHT was less than 0.5; and (3) the ratio of control+OHT to control was not less than 0.5.

The first instance of this experiment showed increased expression of 75 to 200 genes. This number was further refined upon subsequent repetitions of the method.

FIG. 6 summarizes the 27 genes were up-regulated and 9 genes were downregulated in all three MYC induction experiments. This is a significantly greater number of genes than would be expected to be induced based exclusively on fluctuations due biological or technical variability. Several other previously reported MYC targets showed some evidence of regulation but did not meet our strict criterion of 2-fold induction in all three experiments. The complete data set for all of the experiments reported herein is available at the web site http://www.genome.wi.mit.edu/MPR, the teachings of which are incorporated herein by reference.

Significantly, only two of the genes identified in FIG. 6 as putative MYC target genes have been previously reported as downstream MYC targets [ODC (Bello-Fernandez, C. et al., 1993. Proc. Natl. Acad. Sci. USA. 90:7804-7808; Wagner, A. et al., 1993. Cell Growth Diff. 4:879-883) and nucleolin (Greasley, P. et al., 1999. Nucl. Acids Res. 28:446-453)].

### Example 2

Identification of Direct Versus Indirect Targets of MYC

To discriminate between direct and indirect MYC targets, MYC-ER was activated in the presence of cycloheximide (Galaktionov, K. et al., 1996. Nature. 382:511-517; Grandori, C. et al., 1996. EMBO J. 15:4344-4357). By inhibiting protein synthesis, cycloheximide eliminated the possibility that MYC-induced proteins would subsequently modulate a secondary set of genes. Of the 27 genes consistently induced by MYC-ER, 18 genes (68%) were also up-regulated in the presence of cycloheximide, while almost all of the repressed genes (8/9) were also down-regulated under these conditions (FIG. 6). These results suggest that most of the targets identified are likely to be direct targets of MYC.

### Example 3

Target Verification by Northern Blot Analysis

To verify induction by an independent method, six induced target genes were chosen from the set of putative MYC target gene identified in FIG. 6 for Northern blot analysis. In all cases, the Northern blots confirmed the microarray results indicating up-regulation by MYC-ER. For four genes, the same RNA as was used for the microarray measurements was examined for two separate inductions, and for two genes RNA was investigated from an independent MYC-ER induction. As shown in FIGS. 3A-3C, FKBP52, FABP5, PPIF, EIF5A and cyclin D2 follow a similar pattern of expression to that of the known target gene ODC. The ratio of transcript levels in MYC-ER expressing fibroblasts with and without stimulation determined by Northern blot correlated well with the estimates based on the microarrays: 2.3 (Northern, exp. 1)/2.3 (microarray, exp. 1) and 2.2 (Northern, exp. 2)/2.1 (microarray exp. 2) for FKBP52; 1.8/2.0 and 1.4/2.1 for PPIF; 4.1/3.6 for FABP5; 1.8/2.3-3.0 for EIF5A and 3.5/2.2-5.7 for cyclin D2 (FIGS. 3A and B). Thus, the Northern blot data demonstrate an increase in expression in the same range as expected from the microarray results for all of the genes tested.

To ensure that the transcriptional activity of MYC is required for the observed changes in target gene expression, a MYC-ER fusion protein was also tested in which an internal deletion (bp 106-143) renders the protein transcriptionally inactive (Penn, L. et al., 1990. Mol Cell. Biol. 10:4961-4966). As shown in FIG. 3A, neither ODC nor three MYC target genes identified from the microarray analysis were induced by this transcriptionally inactive fusion protein. In addition, p21 was selected as an example of a repressed MYC target (FIG. 3C). Within two hours after OHT stimulation, levels of p21 had decreased.

### Example 4

Altered Expression of Putative MYC Targets During Differentiation.

In order to determine whether the putative targets identified in the microarray assays are influenced by changes in MYC levels under physiologically relevant conditions, it was assessed whether these targets are also affected during the shut-off of endogenous MYC which accompanies hematopoietic differentiation (Henriksson, M. and Luscher, B., 1996. Adv. Cancer Res. 68:109-182 1996). In FIG. 6, ratios of gene expression in differentiated and undifferentiated HL60 cells are given for each of the genes identified as a candidate MYC target in the MYC-ER experiments. Seventeen of the 27 genes consistently induced in the MYC-ER experiments showed a greater than 2-fold decline in expression as HL-60 cells differentiated, while 4 of the 9 genes repressed by MYC-ER increased in abundance more than two-fold. Therefore, genes identified by the conditional induction model discussed above also showed regulation in a physiological context. These findings support the conclusion that the identified genes, which are consistently regulated during both cell cycle progression and differentiation, are MYC target genes.

### Example 5

Identifying Putative MYC Targets in the Myeloid Differentiation Data Alone

Previous reports have suggested that specific transcriptional networks may be identifiable based on analysis of expression data in model systems in the absence of any a priori knowledge. While this approach has yielded success in yeast models, mammalian systems have proven more difficult to decipher. It was determined whether a strategy of defining genes with expression profiles similar to myc in three myeloid differentiation experiments (Tamayo, P. et al., 1999. Proc. Natl. Acad. Sci. USA. 96:2907-2912) would have identified the same genes as the conditional MYC model system. Five of the top ten genes that showed an expression pattern most similar to MYC in the differentiation experiments were independently discovered as MYC targets when MYC itself was overexpressed (binomial p<2×10⁻⁸). This approach was less successful for repressed genes because the genes that increased during cell differentiation were more likely to be cell-type specific.

In summary, the results presented herein indicate that MYC target genes influence a variety of cellular processes including growth, metabolism, cell cycle progression and signal transduction. These results have the potential to provide new connections between MYC and cellular pathways which cannot be anticipated by current knowledge of the molecular mechanisms controlling cellular growth and differentiation.

The relevant portion of all references (e.g., journal articles, books, published patent applications and patents, etc.) and web sites cited herein are incorporated herein by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for inducing the expression of at least one gene selected from group consisting of: AHCY, CCND2, ASS, FKBP52, PBEF, TRAP1, FABP52, GOS2, PPIF, hsRPB8, fibrillarin, TFRC, CksHs2, SLC16A1, IARS, HLA-DRB1, GRPE-homolog, GPI, HSPD1, HDGF, SF2, coup transcription factor, RPS11, EIF5A and EIF4γ in a mammalian cell comprising inducing MYC expression in said cell.

2. The method of claim 1, wherein MYC expression is induced in the cell by transfecting or transducing the cell with a recombinant fusion gene that expresses a chimeric receptor comprising MYC and a ligand binding domain and contacting the resulting cell with a corresponding ligand thereby inducing MYC expression.

3. The method of claim 2, wherein the recombinant fusion gene directs the expression of a fusion protein comprising MYC and the ligand binding domain of the estrogen receptor and wherein the ligand is 4-hydroxytamoxifen.

4. The method of claim 2, wherein the ratio of the expression level observed in cells in the presence of ligand to the expression level observed in cells in the absence of ligand is greater than 2.

5. The method of claim 2, wherein the cell is a primary human cell.

6. A method for repressing the expression of at least one gene selected from group consisting of: AHCY, CCND2, ASS, FKBP52, PBEF, TRAP 1, FABP52, GOS2, PPIF, hsRPB8, fibrillarin, TFRC, CksHs2, SLC16A1, IARS, HLA-DRB1, GRPE-homolog, GPI, HSPD1, HDGF, SF2, coup transcription factor, RPS11, EIF5A and EIF4γ in a mammalian cell comprising inhibiting MYC expression in said cell.

7. A method for causing transcriptional repression of at least one gene selected from the group consisting of: A2M, TPM1, PDGFRA, FN1, CTGF, COL3A1, CDKN1A and a dithiolethione-inducible gene in a mammalian cell comprising inducing MYC expression.

8. The method of claim 7, wherein MYC expression is induced in the cell by transfecting or transducing the cell with a recombinant fusion gene that expresses a chimeric receptor comprising MYC and a ligand binding domain and contacting the resulting cell with an appropriate ligand thereby inducing MYC expression.

9. The method of claim 8, wherein the recombinant fusion gene directs the expression of a fusion protein comprising MYC and the ligand binding domain of the estrogen receptor and wherein the ligand is 4-hydroxytamoxifen.

10. The method of claim 8, wherein the ratio of the expression level observed in cells in the presence of ligand to the expression level observed in cells in the absence of ligand is less than 0.5.

11. The method of claim 7, wherein the cell is a primary human cell.

12. A method for inducing at least one gene selected from the group consisting of: A2M, TPM1, PDGFRA, FN1, CTGF, COL3A1, CDKN1A and a dithiolethione-inducible gene in a mammalian cell comprising inhibiting MYC expression.

13. A method for identifying an agent that regulates MYC-dependent transcriptional regulation of gene expression comprising the steps of:
a) obtaining an indicator cell that expresses a chimeric receptor comprising MYC and a ligand binding domain;
b) contacting the resulting indicator cell with an appropriate ligand in the presence and absence of an agent to be evaluated for its ability to regulate MYC's transcriptional regulation activity;
c) isolating mRNA from a plurality of indicator cells; and
d) comparing the level of gene expression in the indicator cells in the presence or absence of the agent
such that if the effect of MYC on the expression of the gene is enhanced or inhibited in the presence and not the absence of the agent, then the agent regulates MYC-dependent transcriptional regulation of gene expression.

14. The method of claim 13, wherein the agent is tested for its ability to inhibit MYC-dependent transcriptional regulation of gene expression.

15. The method of claim 13, wherein the agent is tested for its ability to activate MYC-dependent transcriptional regulation of gene expression.

16. The method of claim 13, wherein the gene whose level of expression is being evaluated for regulation is selected from the group consisting of: AHCY, CCND2, ASS, FKBP52, PBEF, TRAP1, FABP52, GOS2, PPIF, hsRPB8, fibrillarin, TFRC, CksHs2, SLC16A1, IARS, HLA-DRB1, GRPE-homolog, GPI, HSPD1, HDGF, SF2, coup transcription factor, RPS11, EIF5A and EIF4γ, A2M, TPM1, PDGFRA, FN1, CTGF, COL3A1, CDKN1A and a dithiolethione-inducible gene.

17. The method of claim 13, wherein the chimeric receptor comprises MYC and the ligand binding domain of the estrogen receptor and wherein the ligand that induces c-myc is 4-hydroxytamoxifen.

18. The method of claim 16, wherein the agent is evaluated in the presence of cycloheximide.

19. The method of claim 13, wherein the level of gene expression is determined by hybridization to an oligonucleotide microarray.

20. The method of claim 13, wherein the level of gene expression is determined by Northern blot analysis.

21. A method for treating cell proliferative disorders by altering the transcriptional regulatory activity of MYC in cells.

22. The method of claim 21, wherein the cells are hematopoietic cells.

23. A method for treating cell proliferative disorders by altering MYC expression in cells.

24. The method of claim 23, the cells are hematopoietic cells.

25. A method for detecting cell proliferative disorders comprising the steps of:
a) isolating a cell of interest;
b) determining the level of expression of at least one gene that is regulated by MYC; and
c) comparing the level of expression in the cell of interest and cells that are not characterized as having a proliferative disorder of the gene in step b) such that altered expression of the gene is indicative of a proliferative disorder.

26. The method of claim 25, wherein the isolated cell is a hematopoietic cell.

27. The method of claim 25, wherein the gene in step b) is selected from the group consisting of: AHCY, CCND2, ASS, FKBP52, PBEF, TRAP1, FABP52, GOS2, PPIF, hsRPB8, fibrillarin, TFRC, CksHs2, SLC16A1, LARS, HLA-DRB1, GRPE-homolog, GPI, HSPD1, HDGF, SF2, coup transcription factor, RPS11, EIF5A and EIF4γ, A2M, TPM1, PDGFRA, FN1, CTGF, COL3A1, CDKN1A and a dithiolethione-inducible gene.

28. A method for evaluating anti-proliferative drug candidates comprising the steps of:
a) contacting a cell that conditionally expresses MYC with the anti-proliferative drug candidate;
b) inducing MYC expression;
c) isolating mRNA from the cell; and
d) comparing the level of gene expression of at least one MYC-regulated gene in cells in the presence or absence of the anti-proliferative drug candidate
wherein a difference in expression indicates the effect of the anti-proliferative drug candidate on the transcriptional regulatory activity of MYC.

29. The method of claim 28, wherein the anti-proliferative drug candidate is evaluated in hematopoietic cells.

30. A method for detecting MYC target genes comprising the steps of:
a) inducing MYC expression in an indicator cell;
b) isolating mRNA from induced indicator cells; and
c) comparing the level of gene expression of at least one mRNA transcript in cells induced for MYC expression with the level of gene expression of the mRNA transcript in cells that have not been induced for MYC expression,
wherein altered expression of the gene corresponding to the mRNA transcript in MYC-induced cells indicates the gene is a MYC target gene.

31. The method of claim 30, wherein the level of gene expression is determined using a hybridization assay.

32. The method of claim 31, wherein the hybridization assay comprises a step of contacting cellular mRNA with an oligonucleotide microarray fused to a chip.

33. The method of claim 32, wherein the chip is selected from the group consisting of: Affymetrix HUM6000-1, Affymetrix HUM6000-2, Affymetrix HUM6000-3 and Affymetrix HUM6000-4.

34. A method for inducing the expression of at least one gene selected from group consisting of: AHCY, CCND2, ASS, FKBP52, TRAP1, FABP52, GOS2, PPIF, fibrillarin, TFRC, CksHs2, SLC16A1, ARS, GRPE-homolog, HDGF, and EIF5A in a mammalian cell comprising inducing MYC expression in said cell.

35. A method for causing transcriptional repression of at least one gene selected from the group consisting of: A2M, TPM1, PDGFRA, FN1, CTGF, COL3A1, and CDKN1A in a mammalian cell comprising inducing MYC expression.
